(19) United States
(12) Patent Application Publication
(10) Pub. No.: US 2025/0009550 A1
(43) Pub. Date: Jan. 9, 2025

(54) Excrement collection apparatus and nursing machine

(71) Applicant: SUZHOU ALTON ELECTRICAL & MECHANICAL INDUSTRY CO., LTD., Suzhou City (CN)

(72) Inventors: Weidong LU, Suzhou City (CN); Qiang SONG, Suzhou City (CN); Haibo WANG, Suzhou City (CN)

(21) Appl. No.: 18/218,023

(22) Filed: Jul. 4, 2023

(51) Int. Cl. A61F 5/44; A61F 5/453; A61G 9/00

(52) U.S. Cl. CPC A61F 5/4408; A61F 5/453; A61G 9/00

(57) ABSTRACT

Disclosed are an excrement collection apparatus and a nursing machine. The excrement collection apparatus is applied to the nursing machine and includes a wrapping pant member worn on a human body and a working head detachably combined with the wrapping pant member, the working head includes an excrement pool for accommodating human excrement, the wrapping pant member includes a connecting bracket detachably connected to the working head and an excretion port for human excretion, the connecting bracket is located on a crotch of the wrapping pant member, the connecting bracket is enclosed to form at least a part of the excretion port; in a state that the connecting bracket of the wrapping pant member is butted with the working head, the connecting bracket is attached to the working head, so that the excretion port and the excrement pool are combined together for human excretion.

### TECHNICAL FIELD

The disclosure relates to an excrement collection apparatus and a nursing machine applying the excrement collection apparatus.

### BACKGROUND OF THE INVENTION

A nursing machine is a human excrement collection equipment and is mainly configured for receiving and treating excrement of disabled patients. The nursing machine generally includes an excrement collection apparatus, a main unit, a pipeline apparatus (which generally includes a sewage suction pipeline, a water supply pipeline and the like) connected between the excrement collection apparatus and the main unit, wherein the excrement collection apparatus generally includes a working head for receiving human excrement and a wrapping pant member, the wrapping pant member assists in directly or indirectly wearing the working head on a human body, the working head includes an excrement pool for accommodating the human excrement, the main unit generally includes a suction apparatus, a sewage bucket and a clean water bucket and the like, and when the excrement collection apparatus is worn on the human body for use, the human body excretes into the excrement pool, the suction apparatus sucks, by means of the pipeline apparatus, excrement received by the excrement collection apparatus, and stores the same in the sewage bucket. With regard to the existing excrement collection apparatus, the wrapping pant member and the working head cannot be conveniently combined, so that the use experience of users needs to be improved.

### SUMMARY OF THE INVENTION

Some embodiments of the disclosure provide an excrement collection apparatus with better user experience. The excrement collection apparatus is applied to a nursing machine, and includes a wrapping pant member worn on a human body, and a working head detachably connected to the wrapping pant member, wherein the working head includes an excrement pool for accommodating human excrement, the wrapping pant member includes a connecting bracket detachably connected to the working head, and an excretion port for the excretion of the human body, the connecting bracket is located on a crotch of the wrapping pant member, the connecting bracket is enclosed to form at least a part of the excretion port, and in a state that the connecting bracket of the wrapping pant member is butted with the working head, the connecting bracket is attached to the working head, so that the excretion port is combined with the excrement pool for the excretion of the human body; and the excrement collection apparatus may further include a corresponding locking mechanism, so as to facilitate a locking between the working head and the connecting bracket.

### BRIEF DESCRIPTION OF THE DRAWINGS

FIG. 1 is a stereogram of one angle of an excrement collection apparatus in an embodiment of the disclosure (a first joint apparatus and a second joint apparatus are connected to a pipeline connection side of a working head);

FIG. 2 is a stereogram of another angle of an excrement collection apparatus in an embodiment of the disclosure (a first joint apparatus and a second joint apparatus are connected to a pipeline connection side of a working head);

FIG. 3 is a view of a wearing member in a separated state from a working head in an embodiment of the disclosure (a first joint apparatus and a second joint apparatus are connected to a pipeline connection side of a working head);

FIG. 4 is an exploded view of an excrement collection apparatus in an embodiment of the disclosure (a first joint apparatus and a second joint apparatus are connected to a rear side of a working head);

FIG. 5 is a stereogram of a working head in a state of being butted with a connecting bracket in an embodiment of the disclosure (a first joint apparatus and a second joint apparatus are connected to a pipeline connection side of a working head);

FIG. 6 is a vertical cross-sectional view of FIG. 5;

FIG. 7 is another stereogram of a working head in a state of being butted with a connecting bracket in an embodiment of the disclosure (a first joint apparatus and a second joint apparatus are connected to a pipeline connection side of a working head, and a decorative cover is separated);

FIG. 8 is a partial enlarged view of a working head in a state of being separated from a connecting bracket in an embodiment of the disclosure (a decorative cover is hidden);

FIG. 9 is a view of a working head in a state of being separated from a connecting bracket in an embodiment of the disclosure (a first joint apparatus and a second joint apparatus are connected to a pipeline connection side of the working head, and a decorative cover is separated); and

FIG. 10 is a partial enlarged view of FIG. 6.

The drawings include the following reference signs:
10—wrapping pant member; 100—attachment side; 101—first connection side; 102—excretion port; 103—flexible washer; 1030—second frame opening; 11—working head; 110—front spray head; 111—rear spray head; 112—sewage discharging pipe; 12—excrement pool; 13—second connection side; 131—annular groove; 14—pipeline connection side; 15—first joint apparatus; 16—second joint apparatus; 17—cushion pad; 2—wrapping pant main body; 20—crotch opening; 21—hook and loop fastener; 22—upper wrapping pant piece; 23—lower wrapping pant piece; 24—connecting part; 3—connecting bracket; 30—first frame opening; 31—convex rib; 32—mating portion; 320—head portion; 321—third connecting portion; 33—recess; 34—protruding portion; 35—first U-shaped portion; 350—first portion; 351—second portion; 36—third U-shaped portion; 360—third portion; 361—fourth portion; 37—main body portion; 370—first connecting portion; 371—second connecting portion; 372—second U-shaped portion; 38—decorative cover; 40—first front-end pre-positioning structure; 41—first rear end pre-positioning structure; 42—first teeth; 46—second front-end pre-positioning structure; 47—second rear-end pre-positioning structure; 48—second teeth; 5—locking member; 50—hook portion; 500—first end; 5000—pivot shaft; 501—second end; 51—elastic arm portion; 510—undercut portion; 511—handle; 512—third end; 6—frame portion; 60—third frame opening; 61—left extension arm; 62—right extension arm; 63—cross beam; 64—pivot hole; 70—chassis portion; 71—boss portion; 72—blocking wall; 73—first protruding portion; 74 second protruding portion; 75—rear seat.

### DETAILED DESCRIPTION OF THE INVENTION

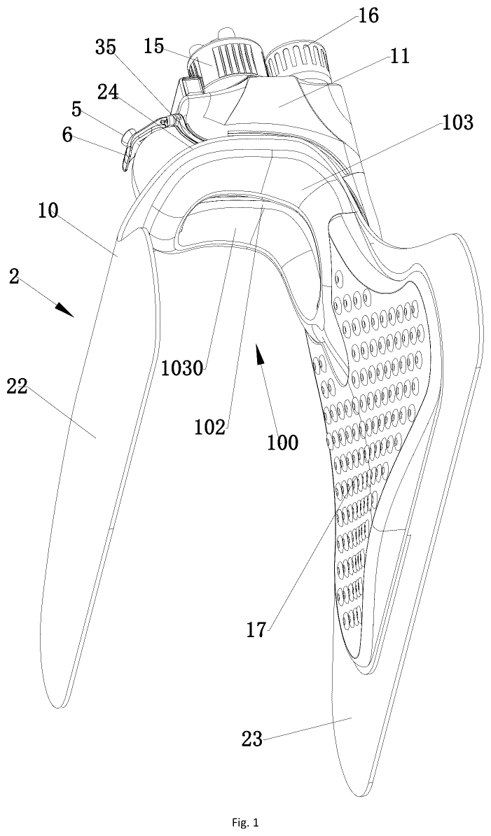

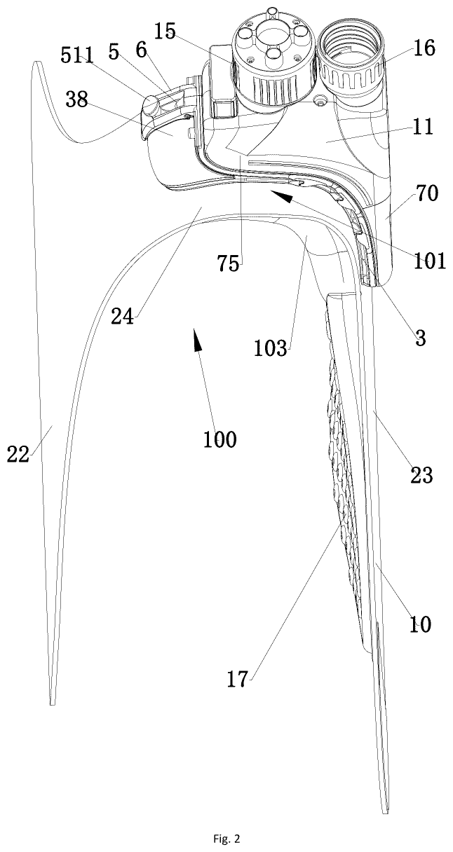

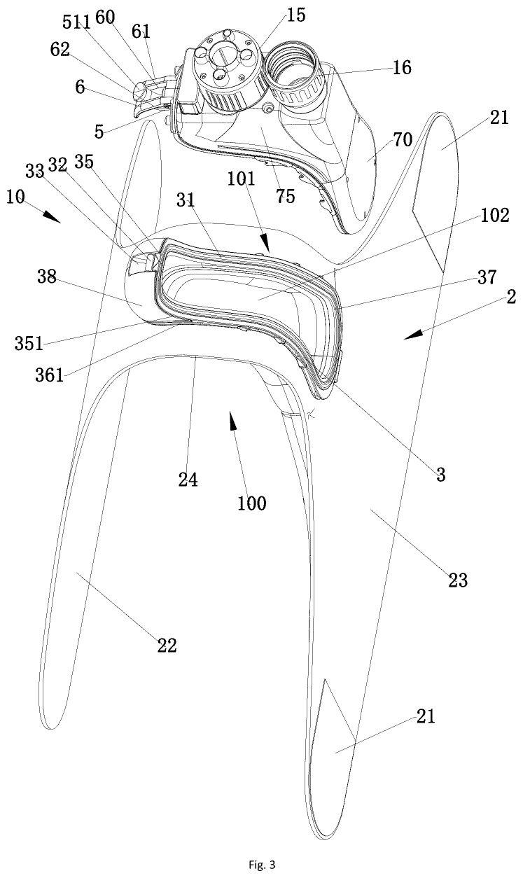

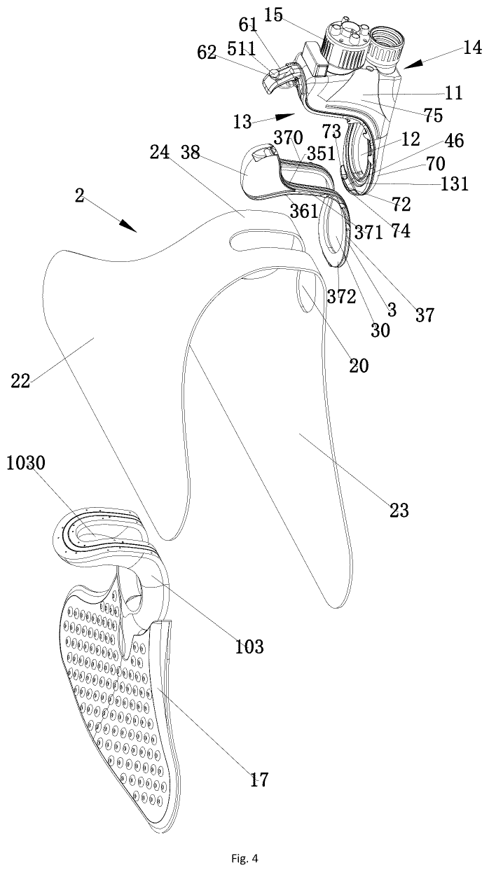

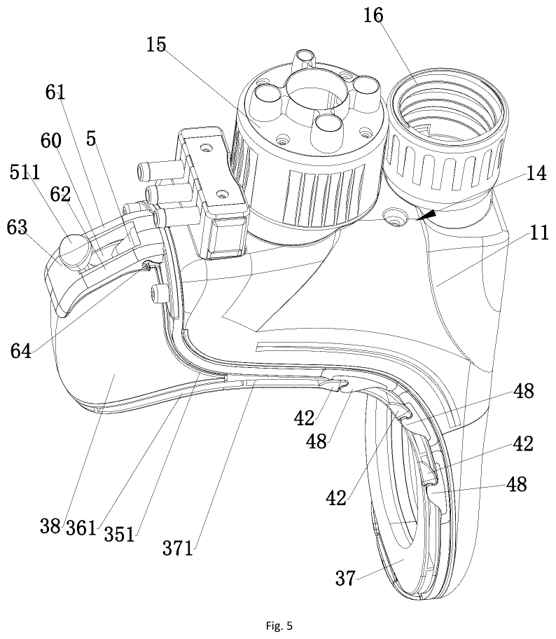

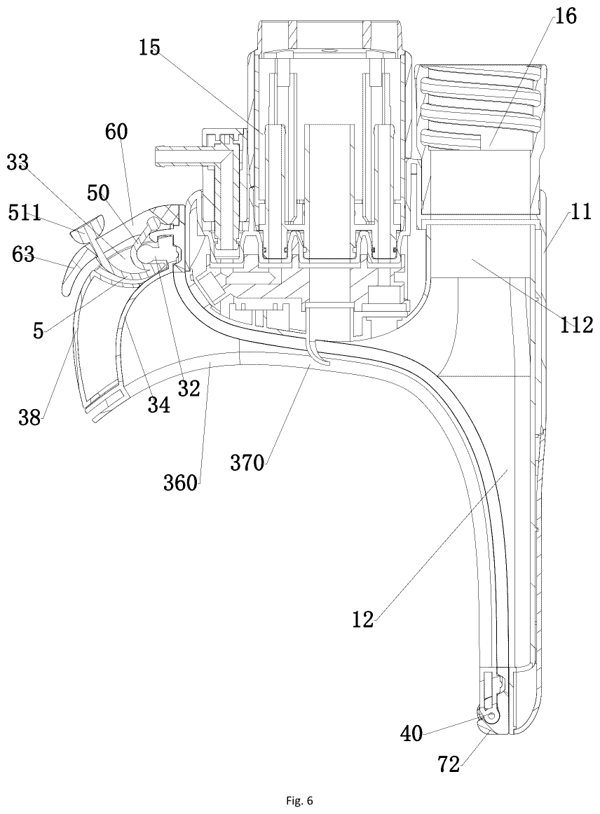

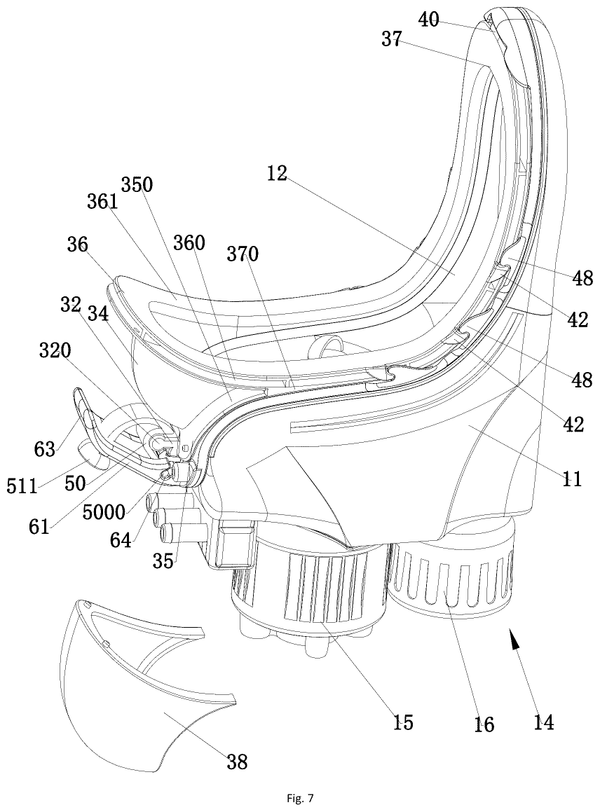

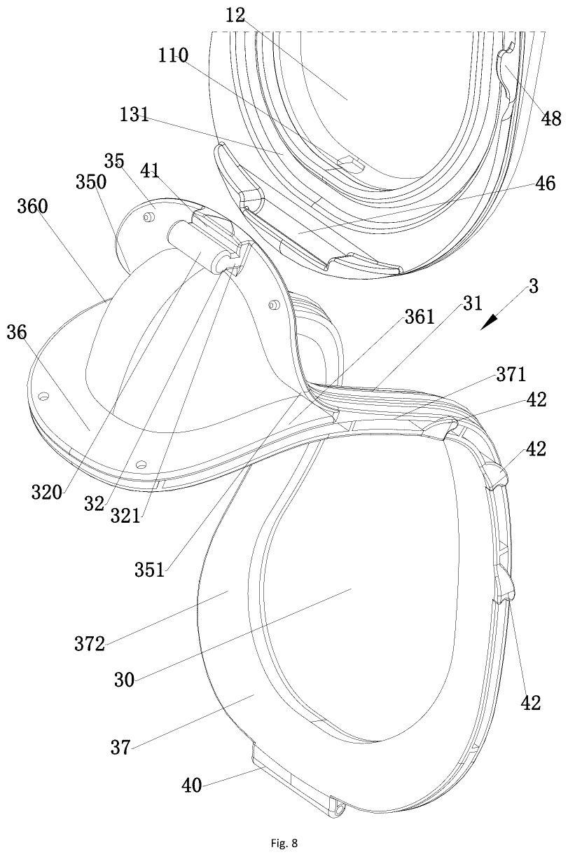

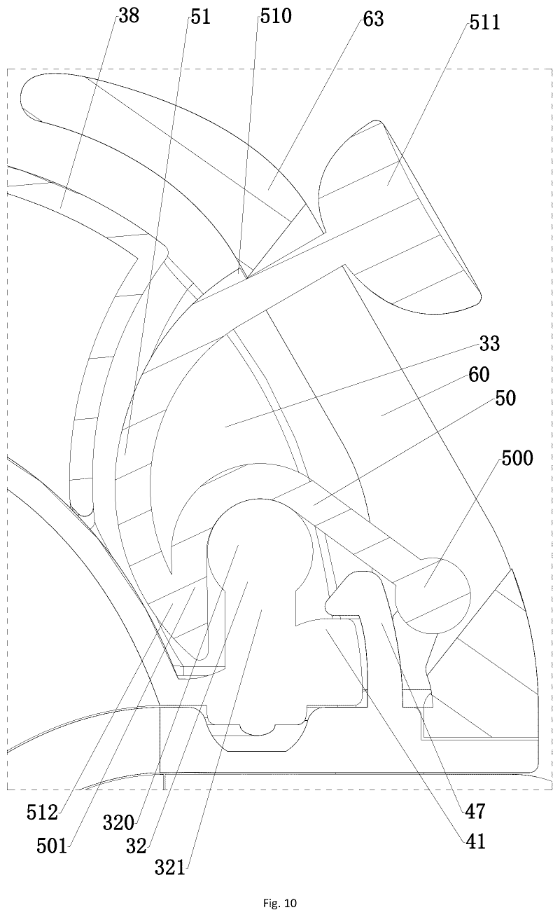

Referring to the embodiments of the disclosure as shown in FIGS. 1-10, disclosed is an excrement collection apparatus, which is applied to a nursing machine. The excrement collection apparatus is worn on a human body for receiving human excrement, the excrement collection apparatus includes a wrapping pant member 10 and a working head 11, and the wrapping pant member 10 is configured for wearing on the human body, the working head 11 is detachably combined with the wrapping pant member 10, and the working head 11 is indirectly worn on the human body by means of the wrapping pant member 10; and the working head 11 includes an excrement pool 12, and the excrement pool 12 is configured for accommodating the human excrement, and the human body excretes into the excrement pool 12.

The wrapping pant member 10 is worn on a crotch of the human body, the wrapping pant member 10 has an attachment side 100 for attaching to the human body, and a first connection side 101 for connecting to the working head 11, the attachment side 100 is opposite to the first connection side 101, and the attachment side 100 and the first connection side 101 form inner and outer sides of the wrapping pant member 10. The wrapping pant member 10 includes an excretion port 102, the excretion port 102 corresponds to the excrement pool 12 for the excretion of the human body, and the excretion port 102 communicates the attachment side 100 with the first connection side 101; after the wrapping pant member 10 is worn on the human body, the anus of the human body faces the excretion port 102, so as to excrete; and after the excretion port 102 is combined with the excrement pool 12, during the excretion of the human body, the excrement is excreted into the excrement pool 12 from the excretion port 102. The wrapping pant member 10 includes a wrapping pant main body 2, the wrapping pant main body 2 includes a crotch opening 20 and a plurality of hook and loop fasteners 21, which are configured for enabling the wrapping pant member 10 to be tightly worn on the human body.

The working head 11 has a second connection side 13 for connecting to the wrapping pant member 10, and a pipeline connection side 14, the excrement pool 12 recesses from the second connection side 13 into the working head 11, the second connection side 13 is configured for connecting to the first connection side 101, the second connection side 13 is opposite to the pipeline connection side 14, and the second connection side 13 and the pipeline connection side 14 form two opposite sides of the working head 11.

The nursing machine further includes a main unit, and a pipeline apparatus connected between the working head 11 and the main unit; the main unit includes wheels, a housing and a motor for providing sewage suction power, and the wheels are located at a bottom of the main unit for moving the main unit; and the motor is located in the housing. A front end of the pipeline apparatus is connected to the pipeline connection side 14 of the working head 11, the pipeline apparatus includes a water supply pipeline and a sewage suction pipeline, and the main unit is able to supply water to the working head 11 by means of the water supply pipeline, so as to flush the human body and the excrement; and under a suction of the main unit, the excrement collected by the excrement pool 12 is conveyed by the sewage suction pipeline to the main unit. The water supply pipeline includes a water supply hose and a first joint apparatus 15 connected to a front end of the water supply hose, the first joint apparatus 15 is detachably butted with the working head 11, the first joint apparatus 15 is connected to the pipeline connection side 14, and the first joint apparatus 15 is butted to the pipeline connection side 14 forwards; the sewage suction pipeline includes a sewage suction hose and a second joint apparatus 16 connected to a front end of the sewage suction hose, the second joint apparatus 16 is detachably butted with the working head 11, the second joint apparatus 16 is connected to the pipeline connection side 14, and the second joint apparatus 16 is butted to the pipeline connection side 14 forwards; and the pipeline connection side 14 of the working head 11 is located on a rear side of the working head 11. The working head 11 includes a front spray head 110 and a rear spray head 111 for flushing the lower body of the human body and/or the excrement, the front spray head 110 is located on a front side of the excrement pool 12, the rear spray head 111 is located on a rear side of the excrement pool 12, and the water supply pipeline is configured for supplying water to the front spray head 110, the rear spray head 111 and the like; and the working head 11 includes a sewage discharging pipe 112, the sewage discharging pipe 112 extends backwards from a rear end of the excrement pool 12, the sewage discharging pipe 112 is butted with the sewage suction pipeline, and the sewage discharging pipe 112 is butted with the second joint apparatus 16.

The wrapping pant member 10 includes a connecting bracket 3, the connecting bracket 3 is detachably connected to the working head 11, the wrapping pant member 10 is connected to the working head 11 by means of the connecting bracket 3, further, the connecting bracket 3 is butted to the second connection side 13 of the working head 11, and the connecting bracket 3 is exposed to the first connection side 101 of the wrapping pant member 10, so as to be connected to the working head 11; since the wrapping pant member 10 is detachably connected to the working head 11 by means of the connecting bracket 3, in a state that the connecting bracket 3 is butted with the working head 11, the connecting bracket 3 is attached to the working head 11, so that the excretion port 102 is combined with the excrement pool 12 for the excretion of the human body; and the connecting bracket 3 is located on a crotch of the wrapping pant member 10, the connecting bracket 3 is in a closed ring, and the connecting bracket 3 surrounds the excretion port 102, the connecting bracket 3 is enclosed to form a first frame opening 30, and the first frame opening 30 forms at least a part of the excretion port 102. The connecting bracket 3 is made of a hard plastic material, and the connecting bracket 3 is adhered to the wrapping pant main body 2, so as to achieve a connection with the wrapping pant main body 2. The working head includes a chassis portion 70 and a rear seat 75, the chassis portion 70 extends forwards from the rear seat 75, the rear seat 75 extends upwards to be higher than the chassis portion 70, the excrement pool 12 is concavely arranged on the chassis portion 70, in the state that the connecting bracket 3 of the wrapping pant member 10 is butted with the working head 11, the chassis portion 70 is located on a lower side of the connecting bracket 3, and the rear seat 75 is located on a rear side of the connecting bracket 3; and in the state that the connecting bracket 3 of the wrapping pant member 10 is butted with the working head 11, the connecting bracket 3 is attached to the chassis portion 70 and the rear seat 75. The front spray head 110 is arranged on the chassis portion 70 and is configured for spraying water into the excrement pool 12; and the rear spray head 111 is arranged on the rear seat 75 and is configured for spraying water towards the excretion port 102, and the rear spray head 111 is located on a front side of the rear seat 75.

The wrapping pant member 10 includes a flexible washer 103, the flexible washer 103 is connected to the wrapping pant main body 2, the flexible washer 103 is enclosed to form a second frame opening 1030, the crotch opening 20 is located between the first frame opening 30 and the second frame opening 1030, and the first frame opening 30, the crotch opening 20 and the second frame opening 1030 are superposed together to form the excretion port 102; the flexible washer 103 is exposed to the attachment side 100 of the wrapping pant member 10, so as to be attached to the human body; the flexible washer 103 is configured for being attached to a periphery of an excretion opening of the human body, and the flexible washer 103 plays a sealing role to prevent the excrement and sewage from leaking from a space between the human body and the wrapping pant member 10, and the flexible washer 103 is selected as a gas flexible washer 103.

After the working head 11 is butted with the connecting bracket 3, the connecting bracket 3 is tightly attached to the second connection side 13 and the excretion port 102 is aligned with the excrement pool 12; the working head 11 further includes a flexible sealing washer and an annular groove 131, the flexible sealing washer is arranged on the second connection side 13, the flexible sealing washer is configured for being attached to the connecting bracket 3 for sealing a space between the connecting bracket 3 and the working head 11, and the connecting bracket 3 includes a convex rib 31 corresponding to the flexible sealing washer; the convex rib 31 is configured for abutting against the flexible sealing washer, so as to ensure a sealing performance after the connecting bracket 3 is connected to the second connection side 13, thereby preventing a leakage of the excrement and sewage generated by flushing; the convex rib 31 is located on a side of the connecting bracket 3 towards the second connection side 13; the flexible sealing washer forms a closed ring, the convex rib 31 forms a closed ring, and the flexible sealing washer surrounds the excrement pool 12; and the annular groove 131 is arranged in the second connection side 13, the flexible sealing washer is arranged in the annular groove 131, the flexible sealing washer is in an annular shape corresponding to the annular groove 131, the annular groove 131 surrounds a periphery of the excrement pool 12, and the rear spray head 111 is located in an area surrounded by the annular groove 131.

The wrapping pant member 10 includes a cushion pad 17, the cushion pad 17 corresponds to a buttocks of the human body, the cushion pad 17 is connected to the wrapping pant main body 2, and the cushion pad 17 is exposed to the attachment side 100 of the wrapping pant member 10 for being attached to the human body, the cushion pad 17 and the flexible washer 103 are configured for being attached to the buttocks of the human body, for jointly buffering the buttocks of the human body, so that the buttocks of the human body uniformly bear a pressure, and thus being more comfortable. The connecting bracket 3 is located between the wrapping pant main body 2 and the working head 11; the connecting bracket 3 is adhered to an outer side surface of the wrapping pant main body 2, the flexible washer 103 is adhered to an inner side surface of the wrapping pant main body 2, and the cushion pad 17 is adhered to the inner side surface of the wrapping pant main body 2. The connecting bracket 3 and the flexible washer 103 are tightly adhered to the surfaces of the wrapping pant main body 2 respectively, so as to achieve a tight connection and sealing with the wrapping pant main body 2. The wrapping pant main body 2 includes an upper wrapping pant piece 22, a lower wrapping pant piece 23 and a connecting part 24, and the connecting part 24 connects the upper wrapping pant piece 22 with the lower wrapping pant piece 23; the crotch opening 20 is formed in the connecting part 24, the connecting bracket 3 is attached to an outer side surface of the connecting part 24, and the flexible washer 103 is attached to an inner side surface of the connecting part 24; and the left and right ends of the upper wrapping pant piece 22 and the left end and the right ends of the lower wrapping pant piece 23 respectively include the plurality of hook and loop fasteners 21. The cushion pad 17 is attached to an inner side surface of the lower wrapping pant piece 23, and the connecting bracket 3 is adhered to the connecting part 24 and is exposed to an outer side of the connecting part 24.

The connecting bracket 3 includes a first front-end pre-positioning structure 40, the chassis portion 70 further includes a second front-end pre-positioning structure 46, the first front-end pre-positioning structure 40 is exposed to the first connection side 101, the second front-end pre-positioning structure 46 is exposed to the second connection side 13, the first front-end pre-positioning structure 40 is embedded with the second front-end pre-positioning structure 46, the first front-end pre-positioning structure 40 is located on a front end of the connecting bracket 3, the second front-end pre-positioning structure 46 is located on a front end of the chassis portion 70, the second front-end pre-positioning structure 46 is in a forward-concave groove shape, and the second front-end pre-positioning structure 46 is able to be sleeved on the first front-end pre-positioning structure 40 backwards; and after the first front-end pre-positioning structure 40 is embedded with the second front-end pre-positioning structure 46, the working head 11 is able to rotate relative to the connecting bracket 3 until the second connection side 13 is attached to the connecting bracket 3 in place. A shape of the second front-end pre-positioning structure 46 is adapted to that of the first front-end pre-positioning structure 40; and the first front-end pre-positioning structure 40 is a partial cylinder extending along a left-right direction, so that the first front-end pre-positioning structure 40 is able to rotate relative to the second front-end pre-positioning structure 46 along with the connecting bracket 3.

The chassis portion 70 includes a boss portion 71, the boss portion 71 is arranged on an upper side of the front end of the chassis portion 70, the second front-end pre-positioning structure 46 is arranged on the boss portion 71, and in the state that the connecting bracket 3 of the wrapping pant member 10 is butted with the working head 11, the boss portion 71 blocks in front of the front end of the connecting bracket 3; and after the second front-end pre-positioning structure 46 is sleeved on the first front-end pre-positioning structure 40 backwards, when the working head 11 is pulled backwards relative to the wrapping pant member 10, the front end of the connecting bracket 3 is able to block the boss portion 71, so as to prevent the working head 11 from separating from the wrapping pant member 10 backwards. The boss portion 71 is located on a front side of a front end of the excrement pool 12, the second front-end pre-positioning structure 46 is a forward-concave groove, the boss portion 71 includes a blocking wall 72 for enclosing the second front-end pre-positioning structure 46, and a first protruding portion 73 and a second protruding portion 74, which are located on the left and right sides of the blocking wall 72, the blocking wall 72 is integrally connected to the first protruding portion 73 and the second protruding portion 74, the first protruding portion 73 and the second protruding portion 74 are separated by the second front-end pre-positioning structure 46, and after the second front-end pre-positioning structure 46 is sleeved on the first front-end pre-positioning structure 40 backwards, when the working head 11 is pulled backwards relative to the wrapping pant member 10, the first front-end pre-positioning structure 40 is able to block the blocking wall 72, so as to prevent the working head 11 from separating from the wrapping pant member 10 backwards.

The connecting bracket 3 further includes a first rear-end pre-positioning structure 41, the first rear-end pre-positioning structure 41 is exposed to the first connection side 101, the rear seat 75 further includes a second rear-end pre-positioning structure 47, the second rear-end pre-positioning structure 47 is exposed to the second connection side 13, and the first rear-end pre-positioning structure 41 is buckled with the second rear-end pre-positioning structure 47. When the working head 11 is assembled on the connecting bracket 3, the second front-end pre-positioning structure 46 is first sleeved on the first front-end pre-positioning structure 40 backwards, and then the working head 11 is rotated relative to the connecting bracket 3 until the second connection side 13 is attached to the connecting bracket 3; and along with the attachment of the working head 11 with the connecting bracket 3, the first rear-end pre-positioning structure 41 is buckled with the second rear-end pre-positioning structure 47. The first rear-end pre-positioning structure 41 is located on a rear end of the connecting bracket 3, and in the embodiment, the second rear-end pre-positioning structure 47 is a hook. By means of an embedding of the first front-end pre-positioning structure 40 with the second front-end pre-positioning structure 46, and a buckling of the first rear-end pre-positioning structure 41 with the second rear-end pre-positioning structure 47, the front end and the rear end of the connecting bracket 3 are preliminarily positioned together with the working head 11, thereby achieving more reliable pre-positioning between the connecting bracket 3 and the working head 11.

The connecting bracket 3 further includes a plurality of first teeth 42, the plurality of first teeth 42 are exposed to the first connection side 101, the working head 11 further includes a plurality of second teeth 48, the plurality of second teeth 48 are exposed to the second connection side 13, the plurality of first teeth 42 are distributed on the left and right sides of the connecting bracket 3, the plurality of second teeth 48 are distributed on the left and right sides of the working head 11, and in a state that the working head 11 is assembled together with the connecting bracket 3, the plurality of first teeth 42 are correspondingly meshed with the plurality of second teeth 48; and if the plurality of first teeth 42 are not able to be correspondingly meshed with the plurality of second teeth 48, it is likely to mean that the connecting bracket 3 is not matched with the working head 11, for example, the connecting bracket 3 is poor in manufacturing and forming, resulting in excessive shrinkage deformation of the connecting bracket 3; and an existence of the plurality of first teeth 42 and the plurality of second teeth 48 is of great significance for ensuring a matching degree of the working head 11 and the connecting bracket 3. The plurality of first teeth 42 are located on the left side and the right side of the connecting bracket 3; the plurality of second teeth 48 are located on the left side and the right side of the second connection side 13; and the plurality of second teeth 48 are located on a periphery of the annular groove 131.

The excrement collection apparatus further includes a locking mechanism, and the locking mechanism is configured for locking the working head 11 with the connecting bracket 3; and the working head 11 is located between two thighs of the human body during using, a space between the two thighs of the human body is narrow, resulting in a difficulty for connecting the working head 11 to the wrapping pant member 10, and a difficulty of an accurate butt joint between the working head 11 and the connecting bracket 3. In the embodiment, after the working head 11 and the connecting bracket 3 are pre-positioned together by means of the above pre-positioning structures, a connecting mechanism is able to be operated more conveniently, so that the working head 11 and the connecting bracket 3 are locked together by means of the locking mechanism, and thus the accurate butt joint between the working head 11 and the connecting bracket 3 is able to be ensured more smoothly.

The locking mechanism includes a mating portion 32 and a locking member 5, the mating portion 32 belongs to a part of the connecting bracket 3, the mating portion 32 is arranged on a first U-shaped portion 35 of the connecting bracket 3, and the locking member 5 is arranged on the rear seat 75; the mating portion 32 is configured to be mated with the locking member 5 in a locking manner, the mating portion 32 is exposed to the first connection side 101, and the mating portion 32 is located on the rear end of the connecting bracket 3; the locking member 5 is rotatable, the locking member 5 is located at a top of the rear seat 75, and a rotation axis of the locking member 5 extends along the left-right direction; and the locking member 5 is able to be buckled on the matching part 32, so as to hook the mating portion 32.

The locking member 5 includes a hook portion 50 and an elastic arm portion 51; the hook portion 50 is integrally connected to the elastic arm portion 51, and the hook portion 50 is arc-shaped; the hook portion 50 has a first end 500 and a second end 501, which are opposite to each other, the first end 500 of the hook portion 50 forms a pivoting connection end of the hook portion 50, and the locking member 5 is rotatably arranged on the top of the rear seat 75 by means of the first end 500 of the hook portion 50; the second end 501 of the hook portion 50 is integrally connected to the elastic arm portion 51; the elastic arm portion 51 is in a cantilever shape, and the elastic arm portion 51 is able to be handheld to rotate the locking member 5; the working head 11 further includes a frame portion 6, the frame portion 6 is arranged on the rear seat 75, the frame portion 6 stands on the top of the rear seat 75, the frame portion 6 obliquely extends forwards and upwards, the locking member 5 is pivotally connected to the frame portion 6, and further, the first end 500 of the hook portion 50 is pivotally connected to the frame portion 6, and the locking member 5 is pivotally connected to the frame portion 6 by means of the first end 500 of the hook portion 50; and the frame portion 6 includes a pivot hole 64, the pivot hole 64 is configured to be pivotally connected to the first end 500 of the hook portion 50, and the first end 500 of the hook portion 50 includes a pivot shaft 5000 adapted to the pivot hole 64, and the pivot shaft 5000 is located on the left and right sides of the first end 500 of the hook portion 50.

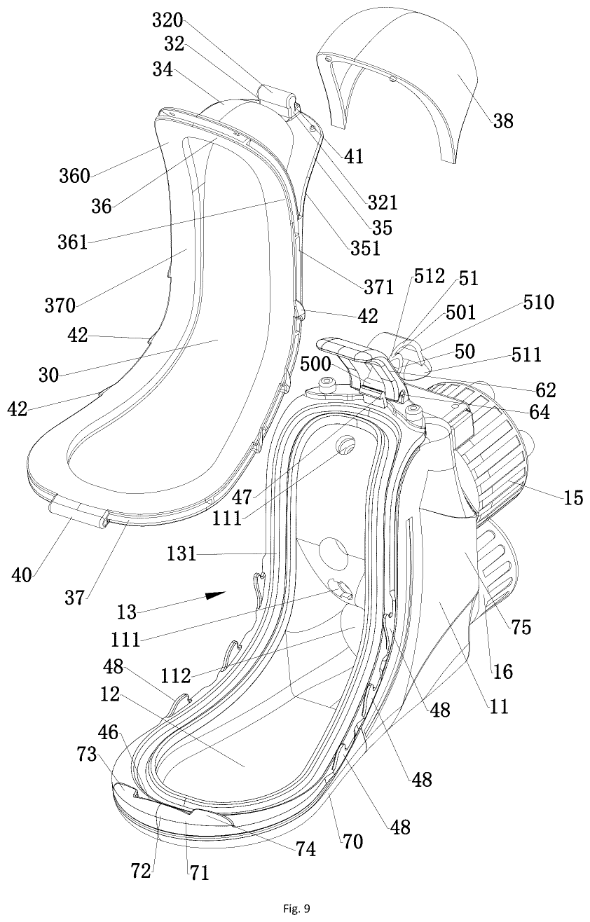

The frame portion 6 is enclosed to form a third frame opening 60, the locking member 5 is able to pass through the third frame opening 60, and the locking member 5 is able to rotate relative to the frame portion 6, so as to enter and exit the third frame opening 60; the elastic arm portion 51 includes an undercut portion 510, the locking member 5 rotates towards the mating portion 32 and is buckled on the mating portion 32, and the undercut portion 510 is configured for abutting against an edge of the third frame opening 60, so as to prevent the locking member 5 from reversing to unbuckle with the mating portion 32; the locking member 5 is able to rotate from a first angle location to a second angle location (as shown in the figures, the locking member 5 is located at the first angle location in FIG. 9, and the locking member 5 is located at the second angle location in other figures), and in a state that the locking member 5 is buckled with the mating portion 32, the locking member 5 is located at the second angle location, and the second angle location belongs to a locking location of the locking member 5; when the locking member 5 is located at the first angle location, the locking member 5 is in an unbuckled state with the mating portion 32; when the locking member 5 is located at the first angle location, the locking member 5 is located on a rear side of the third frame opening 60; and for the hook portion 50, when the locking member 5 is located at the first angle location, the second end 501 of the hook portion 50 is located on the rear side of the third frame opening 60; for the elastic arm portion 51, when the locking member 5 is located at the first angle location, the elastic arm portion 51 is located on the rear side of the third frame opening 60; for the undercut portion 510, when the locking member 5 is located at the first angle location, the undercut portion 510 is located on the rear side of the third frame opening 60; when the locking member 5 is located at the second angle location, the undercut portion 510 is located on a front side of the third frame opening 60; the undercut portion 510 is able to enter and exit the third frame opening 60 along with a rotation of the locking member 5; along with the rotation of the locking member 5 from the first angle location to the second angle location, the second end 501 of the hook portion 50 passes forwards through the third frame opening 60 to rotate towards the mating portion 32, and the hook portion 50 is finally buckled on the mating portion 32; when the locking member 5 is located at the second angle location, the elastic arm portion 51 is partially located in the third frame opening 60, and the elastic arm portion 51 is able to abut upwards against an inner periphery of the third frame opening 60 under an action of an elastic force which is caused by an own elastic deformation of the elastic arm portion 51; the elastic arm portion 51 includes a handle 511 and a third end 512, the third end 512 and the handle 511 of the elastic arm portion 51 form two opposite ends of the elastic arm portion 51, and users are able to hold the handle 511 of the elastic arm portion 51 to rotate the locking member 5, so that the locking member 5 rotates between the first angle location and the second angle location; the third end 512 of the elastic arm portion 51 is integrally connected to the hook portion 50, and further, the third end 512 of the elastic arm portion 51 is integrally connected to the second end 501 of the hook portion 50; and the elastic arm portion 51 is arc-shaped. The second rear-end pre-positioning structure 47 is located on a front side of the first end 500 of the hook portion 50, and the locking member 5 is able to hook the mating portion 32 backwards; when the locking member 5 is located at the second angle location, the locking member 5 covers the second rear-end pre-positioning structure 47; and for the hook portion 50, when the locking member 5 is located at the second angle location, the hook portion 50 covers the second rear-end pre-positioning structure 47. The mating portion 32 includes a head portion 320 and a third connecting portion 321 for connecting the head portion 320, the head portion 320 is in a cylindrical shape extending in a left-right direction, and in the state that the locking member 5 is buckled with the mating portion 32, the locking member 5 is able to be buckled with the head portion 320 of the mating portion 32; the locking member 5 is able to hook the head portion 320 of the mating portion 32 backwards; and the third connecting portion 321 extends upwards, an upper end of the third connecting portion 321 is integrally connected to the head portion 320, and a lower end of the third connecting portion 321 is integrally connected to the first rear end pre-positioning structure 41.

The frame portion 6 includes a left extension arm 61, a right extension arm 62 and a cross beam 63, and the cross beam 63 is connected to the left extension arm 61 and the right extension arm 62; the left extension arm 61 and the right extension arm 62 respectively extend upwards, the cross beam 63 extends in the left-right direction, and the cross beam 63 is connected to an upper end of the left extension arm 61 and an upper end of the right extension arm 62; and the left extension arm 61, the right extension arm 62 and the cross beam 63 are enclosed to form at least a part of the third frame opening 60, the first end 500 of the hook portion 50 is pivotally connected to the left extension arm 61 and the right extension arm 62, and the pivot hole 64 is formed in the left extension arm 61 and the right extension arm 62. When the locking member 5 is located at the second angle location, the undercut portion 510 abuts backwards against the front side of the cross beam 63, so as to prevent the locking member 5 from reversing to unbuckle with the mating portion 32; and when the locking member 5 is located at the second angle location, the elastic arm portion 51 is partially located in the third frame opening 60, and the elastic arm portion 51 abuts against upwards the lower side of the cross beam 63 under an action of the elastic force which is caused by the own elastic deformation of the elastic arm portion 51. The cross beam 63 is located on the top side of the third frame opening 60, the left extension arm 61 and the right extension arm 62 are located on the left and right sides of the third frame opening 60, and the first end 500 of the hook portion 50 is located on the bottom side of the third frame opening 60.

The connecting bracket 3 includes a recess 33, the recess 33 is located on the rear upper portion of the connecting bracket 3, the mating portion 32 is located in the recess 33, the recess 33 is opened backwards, and the locking member 5 is able to enter and exit the recess 33 by means of the rotation relative to the frame portion 6; in the state that the locking member 5 is buckled with the mating portion 32, the connecting bracket 3 of the wrapping pant member 10 and the working head 11 are in a butt joint state; in the state that the locking member 5 is buckled with the mating portion 32, the locking member 5 is partially accommodated in the recess 33; in the state that the locking member 5 is buckled with the mating portion 32, the second end 501 of the hook portion 50 is accommodated in the recess 33; in the state that the locking member 5 is buckled with the mating portion 32, the elastic arm portion 51 is partially accommodated in the recess 33; and in the state that the locking member 5 is buckled with the mating portion 32, the third end 512 of the elastic arm portion 51 is accommodated in the recess 33.

The connecting bracket 3 includes a protruding portion 34, a first U-shaped portion 35, a main body portion 37 and a third U-shaped portion 36, the main body portion 37 includes a first connecting portion 370, a second connecting portion 371 and a second U-shaped portion 372, the first connecting portion 370 and the second connecting portion 371 are located on the left and right sides of the main body portion 37, and the first U-shaped portion 35 is located on the rear side of the third U-shaped portion 36; in the state that the locking member 5 is buckled with the mating portion 32, the first U-shaped portion 35 is attached to the working head 11; the third U-shaped portion 36 is connected to the wrapping pant main body 2; the protruding portion 34 is connected between the first U-shaped portion 35 and the third U-shaped portion 36, the protruding portion 34 and the concave hole 33 are back to back to each other, and the protruding portion 34, the first U-shaped portion 35 and the third U-shaped portion 36 are respectively located on the upper end of the first frame opening 30; and the first U-shaped portion 35 includes a first portion 350 and a second portion 351, the first portion 350 and the second portion 351 are located on the left and right sides of the first U-shaped portion 35, the third U-shaped portion 36 includes a third portion 360 and a fourth portion 361, the third portion 360 and the fourth portion 361 are located on the left and right sides of the third U-shaped portion 36, the first portion 350 and the third portion 360 are connected to form a V shape, and the second portion 351 and the fourth portion 361 are connected to form a V shape. The mating portion 32 is arranged on the first U-shaped portion 35 and is located on the upper side of the first U-shaped portion 35; the first rear-end pre-positioning structure 41 is arranged on the first U-shaped portion 35 and is located on the upper side of the first U-shaped portion 35; and in the embodiment, the first rear-end pre-positioning structure 41 is a boss arranged on the first U-shaped portion 35, and the first rear-end pre-positioning structure 41 is at least partially accommodated in the recess 33.

The first front-end pre-positioning structure 40 is connected to the second U-shaped portion 372, the first front-end pre-positioning structure 40 protrudes forwards from the front end of the second U-shaped portion 372, the main body portion 37 is connected to the first U-shaped portion 35 to form a closed ring, the first connecting portion 370 and the second connecting portion 371 respectively extend upwards from the second U-shaped portion 372, and the second U-shaped portion 372, the first connecting portion 370, the first U-shaped portion 35 and the second connecting portion 371 are sequentially connected to form a closed ring; and in the state that the connecting bracket 3 of the wrapping pant member 10 is butted with the working head 11, the second U-shaped portion 372 is located on the upper side of the chassis portion 70 and is attached to the chassis portion 70, and the first connecting portion 370, the second connecting portion 371 and the first U-shaped portion 35 are respectively attached to the rear seat 75. The main body portion 37 and the third U-shaped portion 36 are connected to form a closed ring, and the second U-shaped portion 372, the first connecting portion 370, the third U-shaped portion 36 and the second connecting portion 371 are sequentially connected to form a closed ring; and in the state that the connecting bracket 3 of the wrapping pant member 10 is butted with the working head 11, the second U-shaped portion 372 is located on the upper side of the chassis portion 70 and is attached to the chassis portion 70, and the first connecting portion 370 and the second connecting portion 371 are respectively located on the front side of the rear seat 75 and are attached to the rear seat 75. The first connecting portion 370, the first portion 350 and the third portion 360 are connected to form a Y shape, the second connecting portion 371, the second portion 351 and the fourth portion 361 are connected to form a Y shape, and the first connecting portion 370 and the second connecting portion 371 respectively extend upwards from the second U-shaped portion 372. The main body portion 37 and the third U-shaped portion 36 are adhered to the wrapping pant main body 2, and the main body portion 37 and the third U-shaped portion 36 is able to be adhered together in various manners such as gluing. The first front-end pre-positioning structure 40 is connected to the second U-shaped portion 372, and the convex rib 31 is arranged on the main body portion 37 and the first U-shaped portion 35.

The main body portion 37 is enclosed to form a part of the first frame opening 30, and the protruding portion 34, the first U-shaped portion 35 and the third U-shaped portion 36 are enclosed at the upper portion of the first frame opening 30. The connecting bracket 3 further includes a decorative cover 38, the decorative cover 38 covers the protruding portion 34, the first U-shaped portion 35 and the third U-shaped portion 36 are attached to an edge of the decorative cover 38, and the recess 33 is formed by recessing a rear side of the decorative cover 38. The second rear-end pre-positioning structure 47 is located between the mating portion 32 and the first end 500 of the hook portion 50; the protruding portion 34 is located in front of the mating portion 32, and in the state that the locking member 5 is buckled with the mating portion 32, the mating portion 32 is located between the protruding portion 34 and the first end 500 of the hook portion 50; and in the state that the locking member 5 is buckled with the mating portion 32, the connected portions of the elastic arm portion 51 and the hook portion 50 are embedded downwards between the protruding portion 34 and the mating portion 32. At least a part of the hook portion 50 and at least a part of the elastic arm portion 51 are connected together to form a tip, and further, the second end 501 of the hook portion 50 and the third end 512 of the elastic arm portion 51 converge together to form an eagle beak shape tip. In the state that the locking member 5 is buckled with the mating portion 32, at least the eagle beak shape tip is embedded downwards between the protruding portion 34 and the mating portion 32.

In the disclosure, a rotation direction of the locking member 5 from the first angle location to the second angle location to achieve buckling with the mating portion 32 is defined as a buckling rotation direction, and a rotation direction which is opposite to the buckling rotation direction and is configured for unlocking the locking member 5 and the mating portion 32 is defined as an unbuckling rotation direction; and in the state that the locking member 5 is buckled with the mating portion 32, when the working head 11 is pulled backwards relative to the connecting bracket 3, the locking member 5 has a tend to rotate in the buckling rotation direction. Therefore, in the state that the locking member 5 is buckled with the mating portion 32, when the working head 11 is pulled backwards relative to the connecting bracket 3, an effect of inhibiting the locking member 5 from rotating in the unbuckling rotation direction is generated, which means that the locking member 5 is not unbuckled with the mating portion 32 when the working head 11 is pulled backwards by an external force, thereby effectively avoiding accidental unbuckling of the locking member 5 and the mating portion 32. In the state that the locking member 5 is buckled with the mating portion 32, contact portions of the locking member 5 and the mating portion 32 are located above the upper side of the rotation axis of the locking member 5, and the hook portion 50 is able to be buckled downwards on the mating portion 32 along with the rotation of the locking member 5; when the locking member 5 rotates relative to the mating portion 32 and is finally buckled in place with the mating portion 32, the rotation direction of the hook portion 50 faces the front lower side; and in the state that the locking member 5 is buckled with the mating portion 32, when the locking member 5 is rotated for unbuckling, the hook portion 50 rotates backwards relative to the mating portion 32, so as to gradually disengage from the mating portion 32. In the state that the locking member 5 is buckled with the mating portion 32, when the working head 11 is pulled backwards relative to the connecting bracket 3, the hook portion 50 pulls the mating portion 32 backwards, at this time, the locking member 5 deforms under the action of a counterforce of the mating portion 32 on the hook portion 50, so as to cause the elastic arm portion 51 to abut against the rear side of the protruding portion 34, the abutment of the protruding portion 34 on the elastic arm portion 51 limits the deformation of the locking member 5 under the action of the external force, which facilitates the structural stability of the locking member 5, and inhibits the accidental unbuckling of the locking member 5 and the mating portion 32.

In the embodiment, the buckling between the first rear-end pre-positioning structure 41 and the second rear-end pre-positioning structure 47 is for the purpose of pre-positioning, thereby facilitating the subsequent operation of the locking member 5 to cooperate with the mating portion 32 and the frame portion 6; as long as the purpose of pre-positioning is met, so the requirement for the buckling stability between the first rear-end pre-positioning structure 41 and the second rear-end pre-positioning structure 47 is very low, in a state that the locking member 5 is unbuckled with the mating portion 32, when the working head 11 is rotated relatively to the connecting bracket 3, the first rear-end pre-positioning structure 41 is able to be unbuckled with the second rear-end pre-positioning structure 47.

The wrapping pant member 10 usually needs to be worn on the human body for a long time, if the working head 11, serving as a relatively heavy object, is connected to the wrapping pant member 10 for a long time and thus is located between the two thighs of the human body, discomfort of the human body is certainly increased; in the embodiment, only the wrapping pant member 10 is worn on the human body and the working head 11 is not connected to the wrapping pant member 10 at regular times (the human body does not excrete); based on the connecting mechanism between the working head 11 and the connecting bracket 3 in the disclosure, the working head 11 is able to be quickly connected to the wrapping pant member 10. Therefore the working head 11 is able to be quickly connected to the wrapping pant member 10 until it is monitored that the user is about to excrete or when the user asks for excretion, the human excrement is still received in time, therefore the human body is able to get rid of the working head 11 at regular times, and thus feels more comfortably.

During assembly, the second front-end pre-positioning structure 46 is first sleeved on the first front-end pre-positioning structure 40 afterwards, and then the first front-end pre-positioning structure 40 and the second front-end pre-positioning structure 46 are used as pivoting connection portions, the first front-end pre-positioning structure 40 is used as an axis of the working head 11, the working head 11 is rotated relative to the connecting bracket 3, until the second connection side 13 is attached to the connecting bracket 3, along with the attachment of the working head 11 and the connecting bracket 3, the first rear-end pre-positioning structure 41 is buckled with the second rear-end pre-positioning structure 47, and along with the attachment of the working head 11 and the connecting bracket 3, the plurality of first teeth 42 are correspondingly meshed with the plurality of second teeth 48; and after the working head 11 and the connecting bracket 3 are pre-positioned together by means of the pre-positioning structures (the first front-end pre-positioning structure 40, the second front-end pre-positioning structure 46, the first rear-end pre-positioning structure 41, and the second rear-end pre-positioning structure 47) described above, the locking member 5 is rotated, so that the locking member 5 is buckled on the mating portion 32. During unbuckling, the handle 511 is able to be pressed down at first to drive the undercut portion 510 of the elastic arm portion 51 to move downwards, so as to deviate from the cross beam 63, and then the locking member 5 is rotated in the unbuckling rotation direction, so that the buckling between the locking member 5 and the mating portion 32 is able to be achieved.

1. An excrement collection apparatus, applied to a nursing machine, comprising:
a working head, comprising an excrement pool for accommodating human excrement; and
a wrapping pant member for being worn on a human body; the wrapping pant member is detachably combined with the working head, and comprises a connecting bracket for being detachably connected to the working head, and an excretion port for an excretion of the human body;
the connecting bracket is located on a crotch of the wrapping pant member, and the connecting bracket is enclosed to form at least a part of the excretion port, and in a state that the connecting bracket of the wrapping pant member is butted with the working head, the connecting bracket is attached to the working head.

2. The excrement collection apparatus according to claim 1, wherein the working head comprises a chassis portion and a rear seat, the chassis portion extends forwards from the rear seat, and the rear seat extends upwards to be higher than the chassis portion, the excrement pool is concavely arranged on the chassis portion; and the connecting bracket comprises a main body portion and a first U-shaped portion, the main body portion comprises a first connecting portion, a second connecting portion and a second U-shaped portion, the second U-shaped portion is located on a front lower part of the connecting bracket and is located at a bottom of the wrapping pant member, the first connecting portion and the second connecting portion are located on the left and right sides of the excretion port, the first connecting portion and the second connecting portion respectively extend upwards from the second U-shaped portion, the first U-shaped portion is located on an upper side of the main body portion, the second U-shaped portion, the first connecting portion, the first U-shaped portion and the second connecting portion are connected in sequence to form a closed ring, in the state that the connecting bracket of the wrapping pant member is butted with the working head, the second U-shaped portion is located on an upper side of the chassis portion and is attached to the chassis portion, the first connecting portion and the second connecting portion are respectively located on a front side of the rear seat, and the first connecting portion, the first U-shaped portion and the second connecting portion are respectively attached to the rear seat.

3. The excrement collection apparatus according to claim 2, wherein the second U-shaped portion, the first connecting portion, the first U-shaped portion and the second connecting portion are connected in sequence to enclose a first frame opening, the wrapping pant member further comprises a wrapping pant main body and a flexible washer to be attached to the human body, the wrapping pant main body comprises a crotch opening, the flexible washer is enclosed to form a second frame opening, the crotch opening is located between the first frame opening and the second frame opening, and the first frame opening, the crotch opening and the second frame opening are superposed together to form the excretion port; in the state that the connecting bracket of the wrapping pant member is butted with the working head, the connecting bracket is located between the wrapping pant main body and the working head; the connecting bracket is located on an outer side of the wrapping pant main body, and the flexible washer is located on an inner side of the wrapping pant main body, the wrapping pant main body further comprises an upper wrapping piece, a lower wrapping piece and a connecting part, the connecting part is connected to the upper wrapping piece and the lower wrapping piece, and the crotch opening is formed in the connecting part; the working head further comprises a front spray head arranged on the chassis portion, a rear spray head arranged on the rear seat, a flexible sealing washer, an annular groove and a sewage discharging pipe, and the front spray head is located on a front side of the excrement pool and is configured for spraying water into the excrement pool; the rear spray head is located on the front side of the rear seat and is configured for spraying water towards the excretion port; the flexible sealing washer is attached to the connecting bracket, so as to seal the connecting bracket and the working head, the flexible sealing washer is arranged in the annular groove, the flexible sealing washer is annular corresponding to the annular groove, the annular groove is arranged in the chassis portion and the rear seat, the annular groove surrounds a periphery of the excrement pool, and the rear spray head is located in an area surrounded by the annular groove; and the sewage discharging pipe extends backwards from a rear end of the excrement pool, and the sewage discharging pipe is butted with a sewage suction pipeline.

4. The excrement collection apparatus according to claim 3, wherein further comprising a locking mechanism for locking between the working head and the connecting bracket, the locking mechanism comprises a mating portion arranged on the first U-shaped portion, and a locking member arranged on the rear seat, and the locking member rotates from a first angle location to a second angle location, so as to be buckled with the mating portion; when the locking member is located at the first angle location, the locking member is in a state of unbuckling with the mating portion; and in a state that the locking member is buckled with the mating portion, the locking member is located at the second angle location.

5. The excrement collection apparatus according to claim 4, wherein the locking member comprises a hook portion for hooking the mating portion, and an elastic arm portion, the elastic arm portion is handheld to rotate the locking member, and the hook portion is buckled downwards on the mating portion along with a rotation of the locking member.

6. The excrement collection apparatus according to claim 5, wherein the locking member is arranged at a top of the rear seat, and a rotation axis of the locking member extends along a left-right direction; the mating portion is arranged on the first U-shaped portion in a protruding state, the hook portion is integrally connected to the elastic arm portion, and the elastic arm portion is in a cantilever shape; and the hook portion comprises a first end and a second end opposite to each other, the elastic arm portion comprises a third end, the third end of the elastic arm portion is integrally connected to the second end of the hook portion, and the second end of the hook portion and the third end of the elastic arm portion converge together to form a tip.

7. The excrement collection apparatus according to claim 6, wherein the first end of the hook portion forms a pivoting connection end of the hook portion, the locking member is rotatably arranged on a top side of the rear seat by means of the first end of the hook portion, and the mating portion is located in front of the first end of the hook portion; the second end of the hook portion and the third end of the elastic arm portion converge together to form the tip with an eagle beak shape; the connecting bracket further comprises a protruding portion, the protruding portion is located in front of the mating portion and is located at a top of the connecting bracket, an edge of the protruding portion is connected to the first U-shaped portion, and in the state that the locking member is buckled with the mating portion, at least the tip with the eagle beak shape is embedded downwards between the protruding portion and the mating portion; and in the state that the locking member is buckled with the mating portion, when the working head is pulled backwards relative to the wrapping pant member, the hook portion pulls the mating portion backwards, and at this time, the locking member deforms under the action of a counterforce of the mating portion on the hook portion, so as to promote the elastic arm portion to abut against a rear side of the protruding portion.

8. The excrement collection apparatus according to claim 5, wherein the locking mechanism further comprises a frame portion arranged on the rear seat, the frame portion stands on a top of the rear seat, the frame portion comprises a left extension arm, a right extension arm, a cross beam and a third frame opening, the cross beam is connected to the left extension arm and the right extension arm, the cross beam is located on a top side of the third frame opening, the left extension arm and the right extension arm are located on a left side and a right side of the third frame opening, the left extension arm, the cross beam and the right extension arm are enclosed to form at least a part of the third frame opening, a rotation axis of the locking member extends along a left-right direction, the locking member rotates relative to the frame portion, so as to enter and exit the third frame opening, and when the locking member is located at the second angle location, the elastic arm portion is partially located in the third frame opening, and the elastic arm portion abuts upwards against a lower side of the cross beam.

9. The excrement collection apparatus according to claim 8, wherein the locking member is pivotally connected to the left extension arm and the right extension arm, the locking member rotates towards the mating portion and is buckled on the mating portion, the elastic arm portion comprises an undercut portion, and when the locking member is located at the second angle location, the undercut portion abuts backwards against a front side of the cross beam, so as to prevent the locking member from reversing to unbuckle with the mating portion.

10. The excrement collection apparatus according to claim 8, wherein the hook portion comprises a first end and a second end opposite to each other, and the first end of the hook portion is pivotally connected to the frame portion; when the locking member is located at the first angle location, the second end of the hook portion is located on a rear side of the third frame opening; and along with a rotation of the locking member from the first angle location to the second angle location, the second end of the hook portion passes forwards through the third frame opening, so as to rotate towards the mating portion, and the hook portion is finally buckled on the mating portion.

11. The excrement collection apparatus according to claim 8, wherein the cross beam extends along a left-right direction; the hook portion comprises a first end and a second end opposite to each other, the first end of the hook portion is pivotally connected to the left extension arm and the right extension arm, and the first end of the hook portion is located on a bottom side of the third frame port; and when the locking member is located at the second angle location, the elastic arm portion is partially located in the third frame opening, and the elastic arm portion abuts upwards against the lower side of the cross beam under an action of an elastic force which is caused by an own elastic deformation of the elastic arm portion.

12. The excrement collection apparatus according to claim 5, wherein a rotation axis of the locking member is located behind the mating portion, and in the state that the locking member is buckled with the mating portion, a contact position between the locking member and the mating portion is located at a front upper side of the rotation axis of the locking member; a rotation direction in which the locking member is rotated from the first angle location to the second angle location to achieve being buckled with the mating portion is defined as a buckling rotation direction, and in the state that the locking member is buckled with the mating portion, when the working head is pulled backwards relative to the connecting bracket, the locking member has a trend of rotating along the buckling rotation direction; at the moment when the locking member rotates relative to the mating portion and is finally buckled in place with the mating portion, a rotation direction of the hook portion is downward; and in the state that the locking member is buckled with the mating portion, when the locking member is rotated to be unbuckled, the hook portion rotates backwards relative to the mating portion, so as to gradually deviate from the mating portion.

13. The excrement collection apparatus according to claim 3, wherein the connecting bracket further comprises a first front-end pre-positioning structure, the first front-end pre-positioning structure is arranged on a front end of the connecting bracket and is located at the bottom of the wrapping pant member, and the first front-end pre-positioning structure is connected to the second U-shaped portion; the chassis portion further comprises a second front-end pre-positioning structure corresponding to the first front-end pre-positioning structure, and the second front-end pre-positioning structure is located on a front end of the chassis portion; and in the state that the connecting bracket of the wrapping pant member is butted with the working head, the first front-end pre-positioning structure is embedded with the second front-end pre-positioning structure.

14. The excrement collection apparatus according to claim 13, wherein the first front-end pre-positioning structure protrudes forwards from a front end of the second U-shaped portion; the second front-end pre-positioning structure is able to be sleeved on the first front-end pre-positioning structure backwards, the chassis portion comprises a boss portion, the boss portion is arranged on an upper side of the front end of the chassis portion, the second front-end pre-positioning structure is arranged on the boss portion, the second front-end pre-positioning structure is a forward-concave groove, and in the state that the connecting bracket of the wrapping pant member is butted with the working head, the boss portion blocks in front of the front end of the connecting bracket; and after the second front-end pre-positioning structure is sleeved on the first front-end pre-positioning structure backwards, when the working head is pulled backwards relative to the wrapping pant member, the front end of the connecting bracket is able to block the boss portion, so as to prevent the working head from separating from the wrapping pant member backwards.

15. The excrement collection apparatus according to claim 1, wherein the working head comprises a chassis portion and a rear seat, the chassis portion extends forwards from the rear seat, the rear seat extends upwards to be higher than the chassis portion, the excrement pool is concavely arranged on the chassis portion, the connecting bracket comprises a main body portion and a third U-shaped portion, the connecting bracket is enclosed to form a first frame opening, the first frame opening is at least a part of the excretion port, and the main body portion is enclosed to form a part of the first frame opening; the wrapping pant member comprises a wrapping pant main body, the main body portion and the third U-shaped portion are adhered to the wrapping pant main body, the main body portion comprises a first connecting portion, a second connecting portion and a second U-shaped portion, the second U-shaped portion, the first connecting portion, the third U-shaped portion and the second connecting portion are connected in sequence to form a closed ring, and in the state that the connecting bracket of the wrapping pant member is butted with the working head, the second U-shaped portion is located on an upper side of the chassis portion and is attached to the chassis portion, and the first connecting portion and the second connecting portion are respectively located on a front side of the rear seat and are attached to the rear seat.

16. The excrement collection apparatus according to claim 15, wherein the connecting bracket further comprises a first U-shaped portion and a protruding portion, the first U-shaped portion is located on a rear side of the third U-shaped portion, the protruding portion is connected between the first U-shaped portion and the third U-shaped portion, the first U-shaped portion comprises a first portion and a second portion, which are located on a left side and a right side of the first U-shaped portion, the third U-shaped portion comprises a third portion and a fourth portion, which are located on a left side and a right side of the third U-shaped portion, the first connecting portion, the first portion and the third portion are connected to form a Y shape, and the second connecting portion, the second portion and the fourth portion are connected to form a Y shape.

17. The excrement collection apparatus according to claim 1, wherein the working head comprises a chassis portion and a rear seat, the excrement pool is concavely arranged on the chassis portion, and in the state that the connecting bracket of the wrapping pant member is butted with the working head, the connecting bracket is attached to the chassis portion and the rear seat; and the connecting bracket surrounds the excretion port, the connecting bracket is enclosed to form a first frame opening, the wrapping pant member comprises a wrapping pant main body and a flexible washer attached to the human body, the wrapping pant main body comprises a crotch opening, the flexible washer is enclosed to form a second frame opening, the crotch opening is located between the first frame opening and the second frame opening, and the first frame opening, the crotch opening and the second frame opening are superposed together to form the excretion port.

18. The excrement collection apparatus according to claim 17, wherein the connecting bracket is adhered to an outer side surface of the wrapping pant main body, and the flexible washer is adhered to an inner side surface of the wrapping pant main body; the wrapping pant main body further comprises an upper wrapping piece, a lower wrapping piece and a connecting part, the connecting part is connected to the upper wrapping piece and the lower wrapping piece, the crotch opening is formed in the connecting part, the connecting bracket and the flexible washer are located on two opposite sides of the connecting portion, and the connecting bracket is adhered to the connecting part and is exposed to an outer side of the connecting portion.

19. The excrement collection apparatus according to claim 1, wherein the working head comprises a chassis portion, a rear seat, a front spray head, a rear spray head, a flexible sealing washer and an annular groove, the working head is indirectly worn on the human body by means of the wrapping pant member, the chassis portion extends forwards from the rear seat, the rear seat extends upwards to be higher than the chassis portion, the excrement pool is concavely arranged on the chassis portion, the front spray head is arranged on the chassis portion, the front spray head is located on a front side of the excrement pool, and the front spray head is configured for spraying water into the excrement pool; the rear spray head is arranged on the rear seat, the rear spray head is located on a front side of the rear seat, and the rear spray head is configured for spraying water towards the excretion port; in the state that the connecting bracket of the wrapping pant member is butted with the working head, the connecting bracket is attached to the chassis portion and the rear seat, the chassis portion is located on a lower side of the connecting bracket, the rear seat is located on a rear side of the connecting bracket, the flexible sealing washer is attached to the connecting bracket for sealing the connecting bracket and the working head, the flexible sealing washer is annular, the flexible sealing washer is arranged in the annular groove, the annular groove surrounds a periphery of the excrement pool, the annular groove is arranged in the chassis portion and the rear seat, and the rear spray head is located in an area surrounded by the annular groove.

20. A nursing machine, comprising an excrement collection apparatus, and the excrement collection apparatus comprises:
a working head, comprising an excrement pool for accommodating human excrement; and
a wrapping pant member, which is worn on a human body, and is detachably combined with the working head, and comprises a connecting bracket detachably connected to the working head, and an excretion port for the excretion of the human body;
the connecting bracket is located on a crotch of the wrapping pant member, and the connecting bracket is enclosed to form at least a part of the excretion port, and in a state that the connecting bracket of the wrapping pant member is butted with the working head, the connecting bracket is attached to the working head.
